(19) United States
(12) Patent Application Publication
(10) Pub. No.: US 2022/0142749 A1
(43) Pub. Date: May 12, 2022

(54) DENTAL IMAGING SYSTEM

(71) Applicant: SMILETRONIX TECHNOLOGIES LTD, CHEADLE HULME (GB)

(72) Inventors: Yashar FARANJANI, Cheadle Hulme, Cheshire (GB); Edward HANSEN, Spanish Fork, UT (US)

(21) Appl. No.: 17/427,567

(22) PCT Filed: Feb. 3, 2020
(86) PCT No.: PCT/EP2020/052548

§ 371 Date: Jul. 30, 2021

(30) Foreign Application Priority Data
Feb. 1, 2019 (EP) 19382076.8

(51) Int. Cl. A61C 9/00; A61B 1/24; A61B 1/04; A61B 1/00; A61B 1/06

(52) U.S. Cl. CPC A61C 9/0053; A61B 1/24; A61B 1/06; A61B 1/00029; A61B 1/04

(57) ABSTRACT

Dental imaging systems are provided for imaging upper-teeth and lower-teeth arches in an oral cavity, the dental imaging system including an outer arch-shaped structure and an inner centralizer structure coupled to each other, and being installable in a predefined position with the upper-teeth and lower-teeth between the outer arch-shaped structure and a camera hub of the inner centralizer structure. The outer arch-shaped structure has outer cameras arranged in such a way that, when the dental imaging system is in predefined position, the outer cameras focus on a front side of both the upper-teeth and lower-teeth. The inner centralizer structure has a support structure and the camera hub, the support structure holding the camera hub and the camera hub including inner cameras arranged in such a way that, when the dental imaging system is in predefined position, the inner cameras focus on a back side of both the upper-teeth and lower-teeth.

This application claims the benefit of European Patent Application EP19382076.8 filed 1 Feb. 2019.

The disclosure relates to dental imaging systems for imaging a dental arrangement including an upper teeth arch and a lower teeth arch in an oral cavity.

### BACKGROUND

Odontology is a medical field that is constantly evolving. Things that were done several years ago are no longer the same. This is because science and technology are advancing very fast. Many dental tools may be found in the field of odontology. Said tools may comprise imaging tools that may be based on e.g. x-ray, dental CT, photography, etc.

Dental imaging may assist the dentist or patient in diagnosis activities. Many times, dental photography permits identifying things that at first sight have been overlooked. For this reason, dental photography is very important and companies are increasingly designing and producing more and more products for this cause.

With dental photography, dental treatments can be tracked very well along the whole cycle of the treatment. Dental images are already part of the patient's medical history, and said photos may help dentists or patients in tracking activities, such as e.g. to be aware of the particular situation of each patient at any time, whenever required.

Dental photography may also help dentists or patients in the planning of cases such as e.g. dental treatments, oral hygiene planning and improvement, etc. Nowadays, suitably planning preventative measures and dental procedures is very important, since good planning may ensure the success of dental treatments.

In some countries, such as e.g. in the United States, dental photography is used as a legal tool that may be very important. Dental photography may permit keeping certain personal registers, files that cannot be modified and that, therefore, may be used as a true proof or the like.

The patent document KR20150021310A discloses a prior art dental imaging system which is illustrated in FIG. 1. This prior art system corresponds to a dental tray 10 comprising a front side wall 11 and a rear side wall 12. The front and rear side walls 11, 12 are formed so that the tray 10 for dental photography surrounds the oral dentition. Multiple camera modules 14 are installed at the front side wall 11 and the rear side wall 12. The front side wall 11 and rear side wall 12 are connected to a u-shaped supporting plate 13. The multiple camera modules 14 comprise a light source 15 and an image sensor 16.

An object of the present disclosure is to provide new dental imaging systems that improve the prior systems aimed at imaging a dental arrangement inside an oral cavity.

### SUMMARY

In an aspect, a dental imaging system is provided for imaging a dental arrangement including an upper teeth arch and a lower teeth arch in an oral cavity of a patient. The dental imaging system includes an outer arch-shaped structure and an inner centralizer structure coupled to each other, and is installable inside the oral cavity in a predefined position with the upper and lower teeth between the outer arch-shaped structure and a camera hub or camera concentrator of the inner centralizer structure.

The outer arch-shaped structure has a plurality of outer cameras arranged on a side of the outer arch-shaped structure in such a way that, when the dental imaging system is installed in the predefined position, the outer cameras focus on a front side of both the upper and lower teeth arches.

The inner centralizer structure has a support structure and the camera hub, the support structure supporting the camera hub which includes a plurality of inner cameras arranged in such a way that, when the dental imaging system is installed in the predefined position, the inner cameras focus on a back side of both the upper and lower teeth arches.

An advantage of the proposed dental imaging system is that fewer cameras may be needed in comparison with prior art dental imaging systems such as the one of FIG. 1. In the proposed dental imaging system, the inner cameras are centralized in a camera hub instead of being distributed along an inner arch-shaped structure such as the inner structure 12 of FIG. 1. The camera hub may have any shape suitable for concentrating the inner cameras as required. The camera hub may have a form of e.g. polyhedron, parallelepiped, sphere, etc. or any combination of them, including flat sides, curved sides, irregular shaped sides, etc. or any combination of them.

Moreover, the prior art system of FIG. 1 is configured to accommodate only one of the teeth arches between outer and inner arch-shaped structures 11, 12. Conversely, the suggested dental imaging system is configured to simultaneously accommodate both upper and lower teeth arches between the outer arch-shaped structure and the inner camera hub or centralizer. This implies that both teeth arches may be simultaneously imaged by the proposed dental imaging system, whereas the prior art system of FIG. 1 requires a multiple-step operation including imaging one of the two teeth arches firstly and the other one secondly. Such a one-step operation of the dental imaging system disclosed herein versus the prior art multiple-step operation necessarily implies that the proposed dental imaging system is operationally more efficient than prior art dental imaging systems.

According to the above considerations, the dental imaging system proposed herein may be cheaper (less cameras) and operationally more efficient (one-step operation) in comparison with prior art systems.

In some examples, the support structure may be configured (e.g. sized, arranged, etc.) in such a way that, when the dental imaging system is installed in the predefined position, the camera hub occupies a substantially central position in the oral cavity or a radially centred location with respect to both upper and lower teeth arches. This central or centred position may be optimal for the inner cameras (at the camera hub) to completely or almost completely capture the back side of both teeth arches with fewer cameras in comparison with prior art systems.

In configurations of the dental imaging system, the support structure may include one or more support connectors having a first and a second end, the one or more support connectors being coupled with the outer arch-shaped structure at the first end and with the camera hub at the second end. The support connector(s) may be formed as or by one or more bodies of any geometrical shape, such as e.g. any polyhedral (elongated) form suitable to implement the holding function of the support connector(s). In some examples, the support connector(s) may have a shape of rod or bar or the like.

In some implementations, the dental imaging system may comprise one or more stopper pieces configured (e.g. arranged, sized, etc.) to guide the installation of the dental imaging system in the predefined position, in which the front side of both the upper and lower teeth arches may be stopped and, therefore, kept by the stopper piece(s) at a predefined distance from the outer cameras.

Such stopper piece(s) may be seen or understood as separator or spacer piece(s) between the outer cameras and the upper and lower teeth arches, so as to keep or retain the front side of both teeth arches at a predetermined distance from the outer cameras. This predefined distance may depend on the technical features of the outer cameras, their distribution along the outer arch-shaped structure, etc. This predetermined distance may be defined longer or shorter depending on the aforementioned aspects of the outer cameras, and the stopper piece(s) may be configured (i.e. shaped, arranged, etc.) accordingly.

Stopper piece(s) may be comprised in the outer arch-shaped structure, in which case stopper piece(s) may be formed as protuberances arranged on same side of the outer arch-shaped structure where the outer cameras are disposed. Additionally or alternatively, stopper piece(s) may be or may comprise protuberances arranged at e.g. an end of the support connector(s) at or near the coupling between the outer arch-shaped structure and the support connector(s).

According to some examples, the dental imaging system may comprise one or more teether pieces configured (e.g. arranged, sized, etc.) to guide the installation of the dental imaging system in the predefined position, which may include the teether piece(s) bitten by the patient and, therefore, arranged or stuck or sandwiched between the upper teeth and the lower teeth of the patient. Taking this into account, the predefined (installation) position may be also denominated as predefined bite position.

Teether piece(s) may be comprised in the outer arch-shaped structure, in which case teether piece(s) may be formed as teether portions protruding from same side of the outer arch-shaped structure where the outer cameras are disposed. Additionally or alternatively, teether piece(s) may be comprised in the inner centralizer structure, in which case teether piece(s) may be or may be comprised in the support connector(s).

Teether piece(s) may be arranged at same or similar height as the outer cameras or, in other words, may be substantially levelled or aligned with respect to the outer cameras. This manner, the outer cameras may result substantially centred or levelled or aligned or equally distanced with respect to the upper teeth arch and to the lower teeth arch, when the dental imaging system is in predefined (bite) position.

In configurations of the dental imaging system, the outer cameras may be arranged in such a way that, when the dental imaging system is installed in the predefined position, respective individual focus scopes of the outer cameras jointly or cooperatively form an overall focus scope completely or almost completely covering the front side of both the upper and lower teeth arches. This technical feature may be implemented by taking into account the technical peculiarities of cameras to be used as outer cameras. Any skilled person should be able to determine quantity, distribution, arrangement, etc. of the outer cameras in order to provide said “outer” overall focus scope depending on their technical characteristics.

In examples of the dental imaging system, the inner cameras may be arranged or centralized in the camera hub in such a way that, when the dental imaging system is in the predefined position, respective individual focus scopes of the inner cameras jointly or cooperatively form an overall focus scope completely or almost completely covering the back side of both the upper and lower teeth arches. This feature may be implemented by taking into account the technical peculiarities of the cameras to be used as inner cameras. Any skilled person should be able to determine quantity, distribution, arrangement, etc. of the inner cameras in order to provide such an overall focus scope depending on their technical characteristics.

According to configurations of the dental imaging system, the outer arch-shaped structure may have an outwardly protruding shape to push lips away from dental imaging zone and, therefore, avoiding lip interferences or disturbances during dental imaging. This may facilitate the operation of the dental imaging system so as to obtain images of the teeth more easily and with “cleaner” conditions.

In implementations of the dental imaging system, the inner centralizer structure may further include a downwardly and/or backwardly protruding shaped piece to push tongue away from dental imaging zone and, therefore, avoiding tongue interferences or disturbances during dental imaging. This downwardly and/or backwardly protruding shaped piece may be coupled to the camera hub or may be a part of the camera hub itself. This feature may also permit facilitating the operation of the dental imaging system to obtain images of the teeth more easily and under less- or non-interfering conditions.

The dental imaging system may be, in some implementations, formed according to a detachable or removable approach such that any component of the dental imaging system is replaceable for hygiene purposes or the like.

The dental imaging system may comprise, in different examples, a battery for powering the inner and outer cameras. This battery may be a rechargeable battery and, in some examples, the dental imaging system may further comprise a wireless charging system for charging the rechargeable battery. This wireless charging system may be e.g. compatible or operable with a pre-existing electric charger, such as e.g. a toothbrush charger.

The dental imaging system may comprise, in various examples, a connection module to connect the dental imaging system with a computing device. This way, images obtained by the dental imaging system are transmittable to the computing device for processing and/or analysis purposes or the like. This connection module may be configured to implement a wireless connection between the dental imaging system and the computing device.

The dental imaging system may also comprise, in diverse implementations, a lighting system configured to provide light in different visible spectrum and/or invisible spectrum wavelengths, so as to capture dental images under different lighting conditions.

The dental imaging system may comprise, in various configurations, a size-adjusting mechanism associated with the outer arch-shaped structure and/or the inner centralizer structure for regulating their size depending on the patient into whom the dental imaging system is to be applied or installed.

In implementations of the dental imaging system, the outer and inner cameras may be arranged in such a way that, when the dental imaging system is installed with the patient's jaw widened, respective individual focus scopes of the outer and inner cameras jointly or cooperatively form an overall focus scope completely or almost completely covering a top side of both the upper and lower teeth.

### BRIEF DESCRIPTION OF THE DRAWINGS

Non-limiting examples of the disclosure will be described in the following, with reference to the appended drawings, in which:

FIG. 1 is a schematic perspective view of a prior art dental imaging system; and

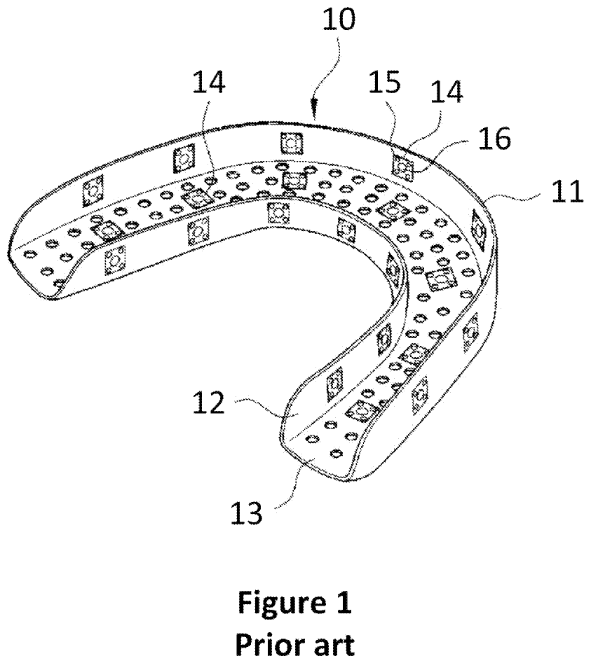

FIG. 2 is a schematic perspective view of an imaging system according to examples.

### DETAILED DESCRIPTION OF EXAMPLES

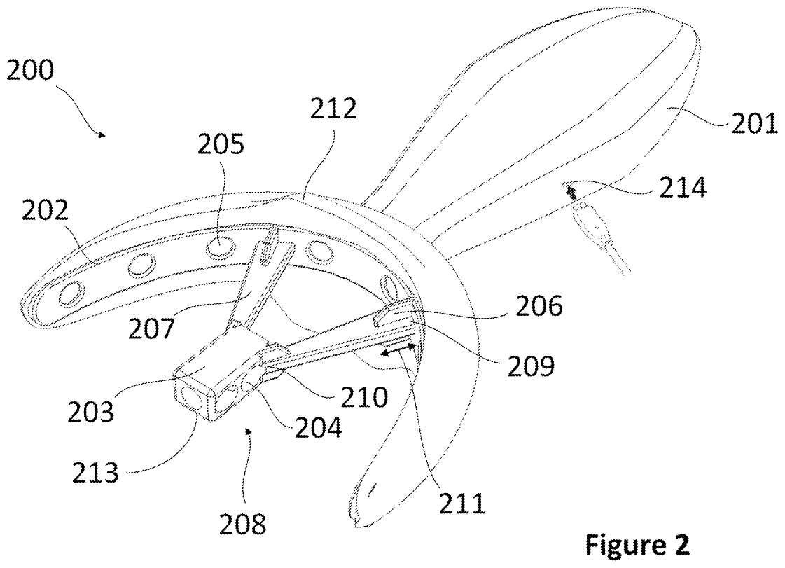

FIG. 2 is a schematic perspective view of imaging systems according to examples. Such dental imaging systems 200 may be used for imaging a dental arrangement including an upper teeth arch and a lower teeth arch in an oral cavity. Dental imaging systems 200 may include an outer arch-shaped structure 202 and an inner centralizer structure 208 coupled to each other. Such dental imaging systems 200 may be installable inside the oral cavity in a predefined position with the upper and lower teeth disposed between the outer arch-shaped structure 202 and an inner camera hub 203 of the inner centralizer structure 208.

The outer arch-shaped structure 202 may have outer cameras 205 arranged on a side of the outer arch-shaped structure 202 in such a way that, when the dental imaging system 200 is installed in the predefined position, the outer cameras 205 focus on a front side of both the upper and lower teeth arch.

The inner centralizer structure 208 may include a support structure 207 and the inner camera hub 203. The support structure 207 may hold or support the inner camera hub 203. The inner camera hub 203 may include a plurality of inner cameras 204 arranged in such a way that, when the dental imaging system 200 is installed in the predefined position, the inner cameras 204 focus on a back side of the upper and lower teeth arch.

The support structure may include one or more support connectors 207 having a first end 209 and a second end 210. The one or more support connectors 207 may be coupled with the outer arch-shaped structure 202 at the first end 209 and with the inner camera hub 203 at the second end 210.

The outer arch-shaped structure 202, when installed in the predefined position, may longitudinally and/or externally surround the patient's upper and lower teeth arches. The outer cameras 205 may be distributed regularly or uniformly or equidistantly along corresponding side of the outer arch-shaped structure 202, so as to jointly or cooperatively focus on a whole or almost whole front side of both the upper and lower teeth arches.

The inner camera hub 203 may be or may be formed as a centralizer or concentrator of the inner cameras 204. The inner camera hub 203, when installed in the predefined position, may occupy a substantially central position in the oral cavity and/or with respect to the (patient's) upper and lower teeth arches. This central position may be e.g. a substantially radially centred position with respect to the upper and lower teeth arches. The inner cameras 204 may be centralized, arranged in the inner camera hub 203 so as to jointly or cooperatively focus on a whole or almost whole back side of the upper and lower teeth arches.

The proposed dental imaging systems 200 may include guiding element(s) 206, 207 to permit or facilitate the installation in a guided manner of the dental imaging system 200 in the predefined position inside patient's oral cavity. Such guiding element(s) may comprise e.g. one or more stopper pieces 206 and/or one or more teether pieces 207.

The one or more stopper pieces 206 may be configured (e.g. arranged, sized, etc.) to guide or assist in the installation of the dental imaging system 200 in the predefined position. Stopper piece(s) 206 may be or may act as a separator or spacer between the outer cameras 205 and the upper and lower teeth arches, in the sense that the outer cameras 205 are kept or retained at a predefined distance 211 from the front side of the upper and lower teeth.

The stopper piece(s) 206 may be comprised in the outer arch-shaped structure 202 and/or the inner centralizer structure 208. In the former case, stopper piece(s) 206 may be formed as protuberances arranged on same side of the outer arch-shaped structure 202 where the outer cameras 205 are disposed. In the latter case, stopper piece(s) 206 may be formed as protuberances arranged at e.g. the first end 209 of the support connector(s) 207, i.e. at or near the coupling 209 between the outer arch-shaped structure 202 and the support connector(s) 207.

The one or more teether pieces 207 may be configured (e.g. arranged, sized, etc.) to also guide or assist in the installation of the dental imaging system 200 in the predefined position. The teether piece(s) 207 may be configured to be bitten by the patient and, therefore, to be arranged or stuck or sandwiched between the upper teeth and the lower teeth. In this sense, the predefined installation position may also be denominated as predefined bite position.

The teether piece(s) 207 may be comprised in the outer arch-shaped structure 202 and/or in the inner centralizer structure 208. In the former case, teether piece(s) 207 may be formed as teether portions protruding from same side of the outer arch-shaped structure 202 where the outer cameras 205 are disposed. In the latter case, teether piece(s) 207 may be or may be comprised in the support connector(s) 207 of the inner centralizer structure 208.

The teether piece(s) 207 may be arranged at same or similar height as the outer cameras 205 or, in other words, the teether piece(s) 207 may be substantially levelled or aligned with respect to the outer cameras 205. This way, when the dental imaging system 200 is in the bite position, the outer cameras 205 may result substantially centred or levelled or aligned or equally distanced with respect to the upper teeth arch and to the lower teeth arch.

The inner centralizer structure 208 may be configured (e.g. sized, arranged, etc.) in such a way that, when the dental imaging system 200 is installed in the predefined position, the inner camera hub 203 results positioned at a substantially central position of the oral cavity.

The outer cameras 205 may be arranged in such a way that, when the dental imaging system 200 is installed in the predefined position, respective individual focus scopes of the outer cameras 205 jointly form an overall focus scope completely or almost completely covering the front side of both the upper and lower teeth arch.

The inner cameras 204 may be arranged, at the inner camera hub 203, in such a way that, when the dental imaging system 200 is installed in the predefined position, respective individual focus scopes of the inner cameras 204 jointly form an overall focus scope completely or almost completely covering the back side of both the upper and lower teeth arch.

Dental imaging system 200 may comprise a lighting system (not shown) configured to provide light in different visible spectrum and/or invisible spectrum wavelengths, so as to allow capturing images under different lighting conditions, depending on e.g. circumstances, type of photos to be produced, purpose of the photos, diagnosis of different conditions, etc. This lighting system may comprise different light sources suitably configured (arranged, sized, etc.). Any relation between light sources and cameras 204, 205 may exist, such as e.g. one-to-one relation, one-to-many, etc.

The outer arch-shaped structure 202 may have an outwardly protruding shape 212 to push lips away from dental imaging zone and, therefore, avoiding lip interferences or disturbances during dental imaging.

The inner centralizer structure 208 may further include a downwardly and/or backwardly protruding shaped piece 213 to push tongue away from dental imaging zone and, therefore, avoiding tongue interferences or disturbances during dental imaging. The downwardly and/or backwardly protruding shaped piece 213 may be coupled to the inner camera hub 203 or may be comprised in the inner camera hub itself 203.

Dental imaging systems 200 according to present disclosure may be formed according to a detachable approach such that any component of the dental imaging system 200 may be replaceable for e.g. hygiene purposes, customizing aims, reparability, etc.

Dental imaging systems 200 may comprise a connection module 214 to connect the dental imaging system 200 with a computing device, so that images obtained by the dental imaging system 200 are transmittable to the computing device for e.g. processing data, image transfer, analysis, diagnosis, and/or recommendation purposes or the like. The connection module may be configured to implement a wireless connection between the dental imaging system 200 and the computing device. Some computing functions may be part of or may be comprised in the connection module 214.

Dental imaging systems 200 may comprise one or more batteries for powering the inner and outer cameras 204, 205, lighting system, connection module 214, etc. The one or more batteries may be rechargeable. Dental imaging systems 200 may further comprise a wireless charging system for charging the one or more rechargeable batteries. The wireless charging system may be compatible with pre-existing electric charger(s), such as e.g. pre-existing electric toothbrush charger(s).

The suggested dental imaging systems 200 may further comprise a handle 201 or similar piece for easy, comfortable, protective and/or efficient manipulation of the dental imaging system 200. The aforementioned connection module 214 may be comprised in said handle 201.

In some examples, dental imaging systems 200 may comprise a size-adjusting mechanism associated with the outer arch-shaped structure 202, the inner centralizer structure 208, etc. for regulating the size of such components depending on each patient into whom the dental imaging system 200 is to be applied or installed. This way, a same dental imaging system 200 may be used with different patients having differently sized oral cavities, teeth arches, etc.

Any of the dental imaging systems 200 described herein may be used to image or photograph teeth with the purpose of assisting individuals and/or dentists in achieving dental diagnosis. The obtained images may be inspected and/or analysed by using systems based on artificial intelligence (AI), machine learning (ML) approaches, or even through visual inspection by a human being properly skilled for that aim. These inspection and/or analysis functions may be performed by computing system(s) comprised in the dental imaging system 200, or by computing system(s) outside the imaging system 200 connectable with each other.

Dental imaging systems 200 may comprise a connection with a recommendation engine configured to produce recommendations about dental treatments, oral hygiene practices, dietary habits, other oral or non-oral health conditions, etc. on users or patients. Alternatively, the recommendation engine may be comprised in the dental imaging system 200. A reporting system may be associated to or comprised in the recommendation engine so as to send messages or notifications to users about said recommendations. This messaging may be performed through e.g. a suitable messaging device or platform associated with or comprised in the dental imaging system 200.

Dental imaging systems 200 according to present disclosure may thus be or may constitute or form (along with e.g. AI-based system, ML-based system, recommendation engine, etc.) a smart system including e.g. self-diagnosis functionalities, self-planning functionalities, self-tracking functionalities, etc.

In some configurations, the arrangement of the outer cameras 205 in/on the outer arch-shaped structure 202 and of the inner cameras 204 in/on the inner camera hub 203 may be such that, when the dental imaging system 200 is installed with patient's jaw widened, a top side of the upper and lower teeth may be imaged by the cameras 204, 205. That is, with such an arrangement, respective individual vision or focus scopes of the inner and outer cameras 204, 205 may jointly or cooperatively form an overall focus scope completely or almost completely covering a top side of both the upper and lower teeth. In some examples, the position of the inner and outer cameras 204, 205 may be adjustable in order to properly move their individual focus scopes. Any position-regulating mechanism may be used to implement said adjustability with one or more degrees of freedom, such as e.g. upwards, downwards, leftwards, rightwards, etc.

Although only a number of examples have been disclosed herein, other alternatives, modifications, uses and/or equivalents thereof are possible. Furthermore, all possible combinations of the described examples are also covered. Thus, the scope of the disclosure should not be limited by particular examples, but should be determined only by a fair reading of the claims that follow.

1. A dental imaging system for imaging a dental arrangement including an upper teeth arch and a lower teeth arch in an oral cavity of a patient, the dental imaging system including an outer arch-shaped structure and an inner centralizer structure coupled to each other, and being installable inside the oral cavity in a predefined position with the upper and lower teeth between the outer arch-shaped structure and a camera hub of the inner centralizer structure, and wherein
the outer arch-shaped structure has a plurality of outer cameras arranged on a side of the outer arch-shaped structure in such a way that, when the dental imaging system is installed in the predefined position, the outer cameras focus on a front side of both the upper and lower teeth arches; and
the inner centralizer structure includes the camera hub and a support structure holding the camera hub, and the camera hub including a plurality of inner cameras arranged in such a way that, when the dental imaging system is installed in the predefined position, the inner cameras focus on a back side of both the upper and lower teeth arches.

2. A dental imaging system according to claim 1, wherein the support structure is configured in such a way that, when the dental imaging system is installed in the predefined position, the inner camera hub is disposed at a substantially central position of the oral cavity.

3. A dental imaging system according to any of claim 1 or 2, wherein the support structure includes one or more support connectors having a first and a second end, the one or more support connectors being coupled with the outer arch-shaped structure at the first end and with the inner camera hub at the second end.

4. A dental imaging system according to any of claims 1 to 3, comprising one or more stopper pieces configured to guide the installation of the dental imaging system in the predefined position, in which the front side of both the upper and lower teeth is kept or retained by the one or more stopper pieces at a predefined distance from the outer cameras.

5. A dental imaging system according to any of claims 1 to 4, comprising one or more teether pieces configured to guide the installation of the dental imaging system in the predefined position, in which the one or more teether pieces are bitten by the patient and therefore arranged or stuck or sandwiched between the upper teeth and the lower teeth of the patient.

6. A dental imaging system according to any of claims 1 to 5, wherein the outer cameras are arranged in such a way that, when the dental imaging system is installed in the predefined position, respective individual focus scopes of the outer cameras jointly form an overall focus scope completely or almost completely covering the front side of both the upper and lower teeth arches.

7. A dental imaging system according to any of claims 1 to 6, wherein the inner cameras are arranged in such a way that, when the dental imaging system is installed in the predefined position, respective individual focus scopes of the inner cameras jointly form an overall focus scope completely or almost completely covering the back side of both the upper and lower teeth arches.

8. A dental imaging system according to any of claims 1 to 7, wherein the outer arch-shaped structure has an outwardly protruding shape to push lips away from dental imaging zone and, therefore, avoiding lip interferences during dental imaging.

9. A dental imaging system according to any of claims 1 to 8, wherein the inner centralizer structure further includes a downwardly and/or backwardly protruding shaped piece to push tongue away from dental imaging zone and, therefore, avoiding tongue interferences during dental imaging; wherein the downwardly and/or backwardly protruding shaped piece is coupled to the inner camera hub or is comprised in the inner camera hub itself.

10. A dental imaging system according to any of claims 1 to 9, formed according to a detachable or removable approach such that any component of the dental imaging system is replaceable.

11. A dental imaging system according to any of claims 1 to 10, comprising a battery for powering the inner and outer cameras or any other electrical component in the dental imaging system; wherein the battery is a rechargeable battery; and wherein the dental imaging system further comprises a wireless charging system for charging the rechargeable battery.

12. A dental imaging system according to any of claims 1 to 11, comprising a connection module to connect the dental imaging system with a computing device, so that images obtained by the dental imaging system are transmittable to the computing device; wherein the connection module is configured to implement a wireless connection between the dental imaging system and the computing device.

13. A dental imaging system according to any of claims 1 to 12, comprising a lighting system configured to provide light in different visible spectrum and/or invisible spectrum wavelengths, so as to capture dental images under different lighting conditions.

14. A dental imaging system according to any of claims 1 to 13, comprising a size-adjusting mechanism associated with the outer arch-shaped structure and/or the inner centralizer structure for regulating their size depending on the patient into whom the dental imaging system is to be applied or installed.

15. A dental imaging system according to any of claims 1 to 14, wherein the outer cameras and inner cameras are arranged in such a way that, when the dental imaging system is installed with patient's jaw widened, respective individual focus scopes of the outer cameras and inner cameras jointly form an overall focus scope completely or almost completely covering a top side of both the upper and lower teeth.
